(19) United States
(12) Patent Application Publication
(10) Pub. No.: US 2006/0216725 A1
(43) Pub. Date: Sep. 28, 2006

(54) Polymer chain reaction apparatus using marangoni convection and polymer chain reaction method using the same

(76) Inventors: You-seop Lee, Gyeonggi-do (KR); Keon Kuk, Gyeonggi-do (KR); Yong-soo Oh, Gyeonggi-do (KR); Su-ho Shin, Gyeonggi-do (KR); Min-soo Kim, Seoul (KR)

Correspondence Address: CANTOR COLBURN, LLP, 55 GRIFFIN ROAD SOUTH, BLOOMFIELD, CT, 06002, US

(21) Appl. No.: 11/219,182

(22) Filed: Sep. 2, 2005

(30) Foreign Application Priority Data
Sep. 15, 2004 (KR) 10-2004-0073920

(51) Int. Cl. C12Q 1/68; C12P 19/34; C12M 1/34

(52) U.S. Cl. 435/6; 435/91.2; 435/287.2

(57) ABSTRACT

A polymerase chain reaction apparatus includes: a substrate; a high-temperature sidewall erected on the substrate; a low-temperature sidewall erected on the substrate and facing the high-temperature sidewall; and a reaction chamber consisting of the substrate, the high-temperature sidewall, and the low-temperature sidewall, wherein a sample contained in the reaction chamber is repetitively thermal-circulated between the high-temperature sidewall and the low-temperature sidewall using Marangoni convection generated by a surface tension gradient resulting from a temperature difference in an interface between the sample and air. The PCR amplification can be automatically accomplished by surface tension flow generated by Marangoni convection resulting from a temperature difference in an interface between the sample and air when a temperature difference between the sidewalls of the chamber is maintained constant. As a result, it is possible to reduce power consumption, simplify the configuration of a temperature control circuit, and reduce the time for a cycle of amplification.

### CROSS-REFERENCE TO RELATED PATENT APPLICATION

This application claims the priority of Korean Patent Application No. 10-2004-0073920, filed on Sep. 15, 2004, in the Korean Intellectual Property Office, the disclosure of which is incorporated herein in its entirety by reference.

### BACKGROUND OF THE INVENTION

1. Field of the Invention

The present invention relates to a polymerase chain reaction (PCR) apparatus using Marangoni convection, and more particularly, to a novel PCR apparatus having an open reaction chamber including a high-temperature sidewall and a low-temperature sidewall erected on a substrate and facing each other, in which the control of heating, cooling, and cycling is not necessary.

2. Description of Related Art

A polymerase chain reaction (PCR) is a reaction used to clone a fragment of a DNA molecule through cyclic heating/cooling to abundantly increase the amount of the fragment. In order to complete a cycle of cloning in a PCR, the temperature of a DNA sample must be changed from T1 to T3, i.e., T1 (denaturing temperature)→T2 (annealing temperature)→T3 (extension temperature).

In a conventional PCR apparatus shown in FIG. 1, a PCR fluid or a biochemical fluid is confined within a chamber, and the temperature of the chamber is accurately adjusted to repeat a cycle consisting of a denaturing process (94° C.), an annealing process (55° C.), and an extension process (72° C.) to facilitate a PCR. A conventional PCR apparatus is advantageous in that its structure is simple, and only the temperature of a heater must be controlled. However, since the heating and cooling should be repeated in the same chamber or the same test tube confining the biochemical fluid, the heating and cooling is unavoidably delayed. Also, complicated circuitry is necessary to accurately control the temperature.

In another conventional PCR apparatus disclosed in U.S. Pat. No. 5,270,183 and shown in FIG. 2, the PCR fluid or the biochemical fluid is continuously pumped through three different temperature areas in a zigzag pattern to generate the PCR. This apparatus is advantageous in that a circuit for controlling the temperature is not necessary. However, the flow path inevitably passes through a T2 area even when the temperature of the fluid can be changed directly from T3 to T1. Therefore, a long flow path should be provided to maintain an accurate temperature profile.

In a conventional PCR apparatus disclosed in Proc. Miniaturized Total Analysis Systems (uTAS 2001), Luisiana State University, Steven A. Soper et al., pp. 459-461, shown in FIG. 3, the direction of the flow path of FIG. 2 is modified such that the PCR fluid or the biochemical fluid continuously flows through three different temperature areas in the shape of concentric circles to facilitate the polymerase chain reaction. Similar to FIG. 2, this apparatus is advantageous in that a circuit for controlling the temperature is not necessary. However, a length of the flow path becomes shortened after every cycle is accomplished. Therefore, a flow rate should be accurately controlled to maintain a proper temperature profile.

In a conventional PCR apparatus disclosed in Krishnan et al., SCIENCE, vol. 298, Oct. 25, 2002, shown in FIG. 4, the PCR is facilitated by a buoyancy flow between a high-temperature plate and a low-temperature plate that are vertically oriented in a sealed or closed chamber. However, it is difficult to generate such buoyancy flow in a miniaturized PCR chamber structured in a micro system such as a lab-on-a-chip because the cubical buoyancy decreases in proportion to the length of the apparatus cubed when the apparatus has a very short length, less than several centimeters or several micrometers, and thus, sufficient buoyancy cannot be obtained. Therefore, a miniaturized PCR system must use another principle instead of the buoyancy flow. According to embodiments of the present invention, a surface tension is used in this regard.

In a conventional PCR apparatus disclosed in U.S. Pat. No. 6,586,233, shown in FIG. 5, the PCR is facilitated by a convective siphon between a high-temperature area and a low-temperature area in a closed or sealed elliptical channel. However, since this apparatus also employs a principle of natural convection caused by a density variation, the cubical buoyancy decreases in proportion to the length of the apparatus cubed when the apparatus has a very short length, less than several centimeters or several micrometers, and thus, sufficient natural convection cannot be obtained.

As described above, in conventional PCR apparatuses, since the chamber containing a DNA buffer solution is heated and cooled in a cyclic manner to amplify a fragment of a DNA molecule, it is difficult to control temperatures, there is high power consumption, and it takes a long time to accomplish the amplification.

In this regard, the present inventors have made many efforts to solve the aforementioned problems, and have found that a PCR amplifier using Marangoni convection can provide many advantages such as reductions in power consumption and amplification time, and simplification of a temperature control circuit because the PCR amplification can be automatically obtained by a surface tension flow generated by a temperature difference in the interface between the fluid and air when both the sidewalls of the chamber are kept in constant temperatures.

### SUMMARY OF THE INVENTION

The present invention provides a novel PCR apparatus using Marangoni convection.

The present invention also provides a PCR method using the PCR apparatus.

The present invention also provides a method of manufacturing the PCR apparatus.

The present invention also provides a lab-on-a-chip and an inkjet spotter including the PCR apparatus.

According to an aspect of the present invention, there is provided a polymerase chain reaction apparatus comprising: a substrate; a high-temperature sidewall erected on the substrate; a low-temperature sidewall erected on the substrate and facing the high-temperature sidewall; and a reaction chamber consisting of the substrate, the high-temperature sidewall, and the low-temperature sidewall, wherein a sample contained in the reaction chamber is repetitively thermal-circulated between the high-temperature sidewall and the low-temperature sidewall using Marangoni convection generated by a surface tension gradient resulting from a temperature difference in an interface between the sample and air.

A polymer chain reaction (PCR) is a method of amplifying a particular DNA region in several tens thousands of or hundreds thousands of times by repeating a DNA synthesis reaction between two kinds of primers interposing the particular DNA region by using a DNA synthesis enzyme in a test tube. Generally, a cycle of the PCR includes denaturing double strands into a single strand, annealing two kinds of primers interposing a target region of the denatured single DNA strand, and extending the primers to produce complementary sequences on the target region.

In the PCR, the denaturing of the double strands is performed at a high temperature of 90° C., and the primer combination and the DNA synthesis are performed at relatively low temperatures of 50˜60° C. and 70˜75° C., respectively. Therefore, a thermal cycler is necessary to perform the PCR.

In the apparatus according to an embodiment of the present invention, Marangoni convection is generated by a surface tension gradient resulting from a temperature gradient in an interface between a reactive fluid and air. As a result, the fluid flows from a high-temperature region to a low-temperature region. Therefore, the apparatus according to the present invention is discriminated from a conventional apparatus using Rayleigh-Benard convection, in which a buoyancy flow from the high-temperature region to the low-temperature region is generated by a density gradient resulting from a temperature difference in the fluid.

The present invention provides the first method and apparatus adopting a principle of Marangoni convection into PCR amplification. The PCR amplification apparatus uses a surface tension flow in a container having an interface (free surface) between the fluid and the air. Therefore, the reaction chamber used in the PCR apparatus according to the present invention is preferably not closed, but is open such that an interface exists between the fluid and air.

The reaction chamber may further include a cover as far as it comprises an interface between the fluid and air. The cover may be combined with the substrate or a sidewall in a single body.

The high-temperature sidewall may be heated to a temperature appropriate to denature the reactive fluid near the high-temperature sidewall. The high-temperature sidewall may be heated by a heater to a constant temperature of 92˜97° C., preferably, 95° C.

The heater may be embodied in various ways such that the high-temperature sidewall can be heated, such as a thin film resistive heater, a heater using a heat exchanger, a radiation heater, and a hot air blasting heater. More preferably, the heater is a thin film heater made of a material selected from a group consisting of platinum, polysilicon, and tantal aluminum. Also, the heater may be installed in the inside or outside of the sidewall, and a sensor for adjusting the temperature may be provided with the heater.

The low-temperature sidewall may be cooled to a temperature appropriate to anneal the reactive fluid near the low-temperature sidewall. The low-temperature sidewall may be cooled by a cooler to a constant temperature of 44˜56° C., preferably, 50° C.

The cooler may be embodied in various ways such that the sidewall can be cooled, such as a cooling fan or a thermal cycler. Preferably, the cooler may be a Peltier device. The cooler may be installed in the inside or outside of the low-temperature sidewall, and a sensor for adjusting the temperature may be provided together with the cooler.

The sample experiences DNA denaturing near the high-temperature sidewall, and the fluid at the surface of the reactive fluid rapidly flows to the low-temperature sidewall due to Marangoni convection, where the fluid is annealed. Then, a lower region of the reactive fluid slowly flows from the low-temperature sidewall to the high-temperature sidewall, thereby generating extension, i.e., the synthesis of new DNA strands.

A gap between the high-temperature sidewall and the low-temperature sidewall may be 2 mm to 3 cm. In the conventional PCR apparatus using a buoyancy flow, since the buoyancy is proportional to the volume of a container, a driving force decreases as the size of the container decreases. However, in the PCR apparatus according to the present invention, since the surface tension is proportional to the area of the fluid surface, a sufficient driving force can be obtained even when the container is small. Therefore, the PCR apparatus according to an embodiment of the present invention can be embodied in a DNA chip, a subminiature DNA detector, or a lap-on-a-chip.

The reaction chamber may be made of a material selected from a group consisting of glass, quartz, silicon, plastic, polymer, ceramic, and metal. Preferably, the reaction chamber is made by etching a silicon wafer using photolithography, i.e., a typical semiconductor manufacturing process.

The reaction chamber may have an optical detection window. Through the optical detection window, the PCR reaction in the chamber can be optically detected using a conventional PCR detection method in a real-time manner.

According to another aspect of the present invention, there is provided a polymerase chain reaction method comprising: putting a sample into the reaction chamber of any one of the above-described polymerase chain reaction apparatuses, maintaining temperatures of the high-temperature sidewall and the low-temperature sidewall constant; and repetitively thermal-circulating the sample contained in the reaction chamber between the high-temperature sidewall and the low-temperature sidewall using Marangoni convection.

The sample may include a typical template DNA, oligonucleotide primers, four dNTP's (i.e., dATP, dCTP, dGTP, and dTTP), a thermostable DNA polymerase, and a reactive buffer.

The high-temperature sidewall may be maintained at a constant temperature of 92˜97° C., appropriate for denaturing the DNA sample, and the low-temperature sidewall may be maintained at a constant temperature of 48˜54° C., appropriate for annealing the sample.

The sample may contain a fluorescent material to detect amplification of a nucleic acid in a real-time manner. The amplification sample may be a plasmid DNA. A driving fluid may be produced by adding primers, dNDP, a base, and a buffer solution containing a DNA polymerization enzyme into an initial sample. Distinct amplification DNA bands are visible when the fluorescent material in the DNA sample that has been amplified using the PCR reaction is detected.

According to still another aspect of the present invention, there is provided a method of manufacturing a PCR apparatus including a photolithographic process. In the method, photolithography is applied to a first substrate to form the pattern of a flow path and a PCR chamber on its upper surface. The first substrate may be made of a material selected from a group consisting of silicon, glass, polycarbonate, polydimethylsiloxane, and polymethylmetaacrylate. The first substrate may be etched to have a desired thickness by wet etching or dry etching such as a reactive ion etching. If necessary, the photolithographic process and the etching process may be repeated several times to allow the flow path and the chamber to have varying depths. A hydrophobic treatment is applied to the upper portion of a second substrate, which is a cover for preventing evaporation of the DNA reaction fluid in the PCR chamber to resist wetting. After patterns of an inlet and an outlet for the sample are formed on the first substrate through photolithography, the inlet and the outlet are finished by sound-blasting. If it is necessary to form an electrode structure on the second substrate, electrode patterns are formed through photolithography and are obtained using a lift-off procedure. Subsequently, the first and second substrates are bonded using a method such as anodic bonding, fluorine bonding, thermal bonding, or polymer film bonding.

According to still another aspect of the present invention, there is provided a lab-on-a-chip comprising any one of the above-described polymerase chain reaction apparatuses and an electrophoresis performing unit connected to the polymerase chain reaction apparatus in a fluidic manner.

When the sample in the chip passes through the PCR apparatus, the DNA is amplified. When the sample passes through the electrophoresis performing unit, the DNA is separated depending on its molecular amount or charge to detect target DNA.

The substrates may be made of a material selected from a group consisting of glass, quartz, silicon, plastic, polymer, ceramic, or metal. The electrophoresis unit may have multiple channels performing capillary electrophoresis. The PCR amplification apparatus and the electrophoresis performing unit may be formed on a substrate through photolithography.

According to still another aspect of the present invention, there is provided an inkjet spotter comprising: any one of the above-described polymerase chain reaction apparatuses formed on a substrate; a restrictor connected to the polymerase chain reaction apparatus in a fluidic manner; an ejecting chamber storing a DNA solution from the polymerase chain reaction apparatus via the restrictor; an ejecting driving element providing a driving force of the DNA solution ejection; and a nozzle ejecting the DNA solution from the ejecting chamber.

The inkjet spotter according to the present invention may be similar to a typical inkjet spotter used to manufacture a conventional DNA micro-array except for a PCT apparatus. The ejecting driving element may be a thermal type (similar to that disclosed in U.S Pat. No. 4,438,191), a Piezo type (similar to that disclosed in U.S. Pat. No. 5,748,214), or an electric field type (similar to that disclosed in U.S. Pat. No. 4,752,783).

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic diagram of a conventional polymerase chain reaction (PCR) apparatus, in which a PCR reaction is facilitated by controlling a temperature of a PCR fluid or a biochemical fluid confined within a chamber (T1=94° C., T2=55° C., and T3=72° C.);

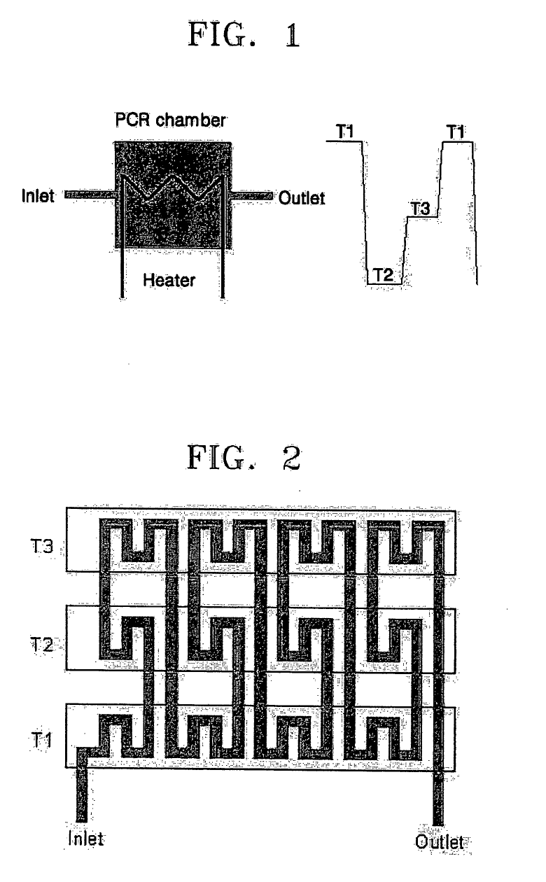

FIG. 2 is a schematic diagram of a conventional PCR apparatus in which a PCR reaction is facilitated by continually pumping a PCR fluid or a biochemical fluid through different temperature regions in a zigzag pattern;

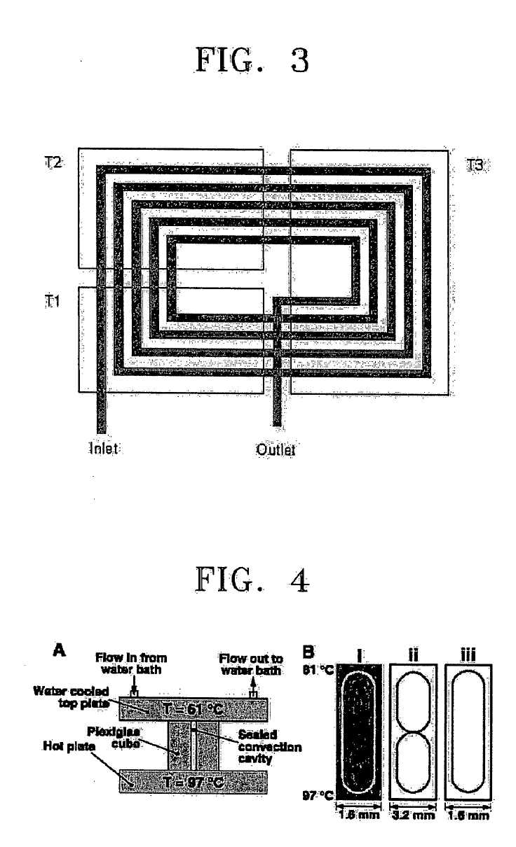

FIG. 3 is a schematic diagram of a conventional PCR apparatus in which a PCR reaction is facilitated by continually pumping a PCR fluid or a biochemical fluid through different temperature regions in concentric circles;

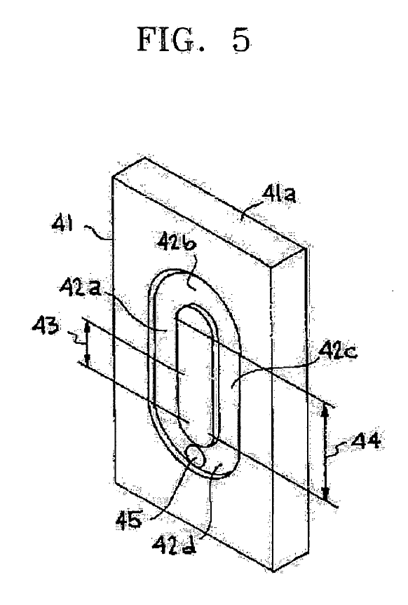

FIG. 4 is a schematic diagram of a conventional PCR apparatus (a Rayleigh-Benard convection cell), in which a PCR reaction is facilitated by a buoyancy flow between a high-temperature plate and a low-temperature plate in a closed or sealed container;

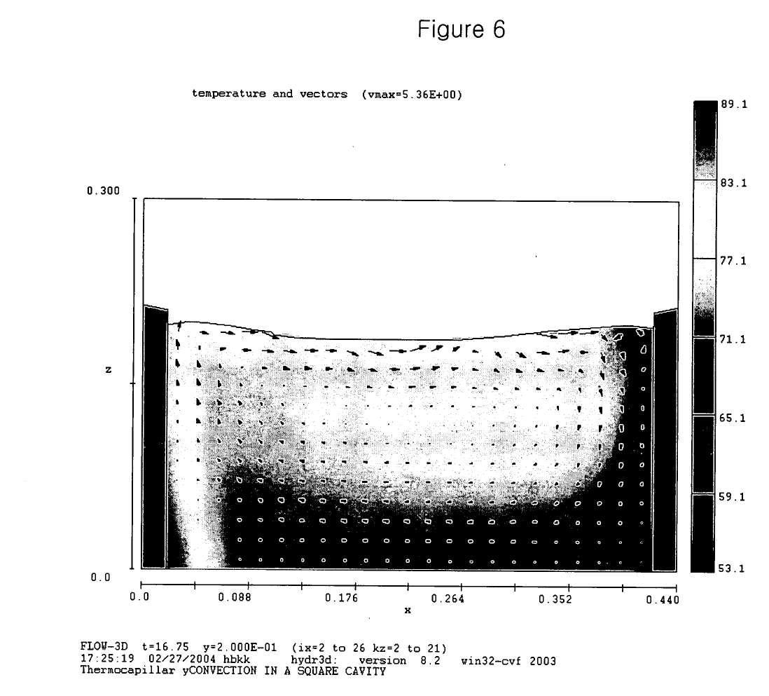

FIG. 5 is a schematic diagram of a conventional PCR apparatus in which a PCR reaction is facilitated by a convective siphon between a high-temperature plate and a low-temperature plate in a closed or sealed elliptical channel;

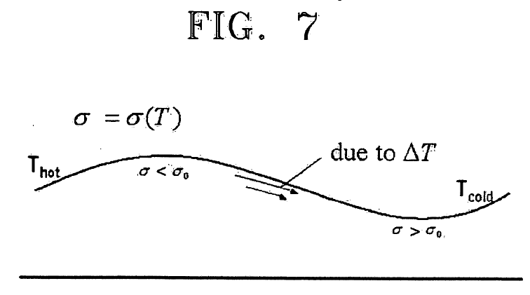

FIG. 6 is a schematic diagram of a PCR apparatus using Marangoni convection according to an embodiment of the present invention;

FIG. 7 is a schematic diagram illustrating a principle of generating capillary tube flow depending on a surface tension gradient at an interface between a fluid and air;

FIG. 8 is a temperature vs. time graph for a PCR reaction process according to an embodiment of the present invention;

FIG. 9 illustrates the result of a flow analysis in a two-dimensional container depending on the speed and the temperature;

FIG. 10 illustrates the result of a flow analysis in a three-dimensional container depending on the speed and the temperature; and

FIG. 11 illustrates an exemplary inkjet spotter according to an embodiment of the present invention.

### DETAILED DESCRIPTION OF THE INVENTION

According to an aspect of the present invention, there is provided a polymerase chain reaction apparatus comprising: a substrate; a high-temperature sidewall erected on the substrate; a low-temperature sidewall erected on the substrate and facing the high-temperature sidewall; and a reaction chamber consisting of the substrate, the high-temperature sidewall, and the low-temperature sidewall, wherein a sample contained in the reaction chamber is repetitively thermal-circulated between the high-temperature sidewall and the low-temperature sidewall using Marangoni convection generated by a surface tension gradient resulting from a temperature difference in an interface between the sample and air.

A polymer chain reaction (PCR) is a method of amplifying a particular DNA region in several tens thousands of or hundreds thousands of times by repeating a DNA synthesis reaction between two kinds of primers interposing the particular DNA region by using a DNA synthesis enzyme in a test tube. Generally, a cycle of the PCR includes denaturing double strands into a single strand, annealing two kinds of primers interposing a target region of the denatured single DNA strand, and extending the primers to produce complementary sequences on the target region.

In the PCR, the denaturing of the double strands is performed at a high temperature of 90° C., and the primer combination and the DNA synthesis are performed at relatively low temperatures of 50˜60° C. and 70˜75° C., respectively. Therefore, a thermal cycler is necessary to perform the PCR.

In the apparatus according to an embodiment of the present invention, Marangoni convection is generated by a surface tension gradient resulting from a temperature gradient in an interface between a reactive fluid and air. As a result, the fluid flows from a high-temperature region to a low-temperature region. Therefore, the apparatus according to the present invention is discriminated from a conventional apparatus using Rayleigh-Benard convection, in which a buoyancy flow from the high-temperature region to the low-temperature region is generated by a density gradient resulting from a temperature difference in the fluid.

The present invention provides the first method and apparatus adopting a principle of Marangoni convection into PCR amplification. The PCR amplification apparatus uses a surface tension flow in a container having an interface (free surface) between the fluid and the air. Therefore, the reaction chamber used in the PCR apparatus according to the present invention is preferably not closed, but is open such that an interface exists between the fluid and air.

The reaction chamber may further include a cover as far as it comprises an interface between the fluid and air. The cover may be combined with the substrate or a sidewall in a single body.

The high-temperature sidewall may be heated to a temperature appropriate to denature the reactive fluid near the high-temperature sidewall. The high-temperature sidewall may be heated by a heater to a constant temperature of 92˜97° C., preferably, 95° C.

The heater may be embodied in various ways such that the high-temperature sidewall can be heated, such as a thin film resistive heater, a heater using a heat exchanger, a radiation heater, and a hot air blasting heater. More preferably, the heater is a thin film heater made of a material selected from a group consisting of platinum, polysilicon, and tantal aluminum. Also, the heater may be installed in the inside or outside of the sidewall, and a sensor for adjusting the temperature may be provided with the heater.

The low-temperature sidewall may be cooled to a temperature appropriate to anneal the reactive fluid near the low-temperature sidewall. The low-temperature sidewall may be cooled by a cooler to a constant temperature of 44˜56° C., preferably, 50° C.

The cooler may be embodied in various ways such that the sidewall can be cooled, such as a cooling fan or a thermal cycler. Preferably, the cooler may be a Peltier device. The cooler may be installed in the inside or outside of the low-temperature sidewall, and a sensor for adjusting the temperature may be provided together with the cooler.

The sample experiences DNA denaturing near the high-temperature sidewall, and the fluid at the surface of the reactive fluid rapidly flows to the low-temperature sidewall due to Marangoni convection, where the fluid is annealed. Then, a lower region of the reactive fluid slowly flows from the low-temperature sidewall to the high-temperature sidewall, thereby generating extension, i.e., the synthesis of new DNA strands.

A gap between the high-temperature sidewall and the low-temperature sidewall may be 2 mm to 3 cm. In the conventional PCR apparatus using a buoyancy flow, since the buoyancy is proportional to the volume of a container, a driving force decreases as the size of the container decreases. However, in the PCR apparatus according to the present invention, since the surface tension is proportional to the area of the fluid surface, a sufficient driving force can be obtained even when the container is small. Therefore, the PCR apparatus according to an embodiment of the present invention can be embodied in a DNA chip, a subminiature DNA detector, or a lap-on-a-chip.

The reaction chamber may be made of a material selected from a group consisting of glass, quartz, silicon, plastic, polymer, ceramic, and metal. Preferably, the reaction chamber is made by etching a silicon wafer using photolithography, i.e., a typical semiconductor manufacturing process.

The reaction chamber may have an optical detection window. Through the optical detection window, the PCR reaction in the chamber can be optically detected using a conventional PCR detection method in a real-time manner.

According to another aspect of the present invention, there is provided a polymerase chain reaction method comprising: putting a sample into the reaction chamber of any one of the above-described polymerase chain reaction apparatuses, maintaining temperatures of the high-temperature sidewall and the low-temperature sidewall constant; and repetitively thermal-circulating the sample contained in the reaction chamber between the high-temperature sidewall and the low-temperature sidewall using Marangoni convection.

The sample may include a typical template DNA, oligonucleotide primers, four dNTP's (i.e., dATP, dCTP, dGTP, and dTTP), a thermostable DNA polymerase, and a reactive buffer.

The high-temperature sidewall may be maintained at a constant temperature of 92˜97° C., appropriate for denaturing the DNA sample, and the low-temperature sidewall may be maintained at a constant temperature of 48˜54° C., appropriate for annealing the sample.

The sample may contain a fluorescent material to detect amplification of a nucleic acid in a real-time manner. The amplification sample may be a plasmid DNA. A driving fluid may be produced by adding primers, dNDP, a base, and a buffer solution containing a DNA polymerization enzyme into an initial sample. Distinct amplification DNA bands are visible when the fluorescent material in the DNA sample that has been amplified using the PCR reaction is detected.

According to still another aspect of the present invention, there is provided a method of manufacturing a PCR apparatus including a photolithographic process. In the method, photolithography is applied to a first substrate to form the pattern of a flow path and a PCR chamber on its upper surface. The first substrate may be made of a material selected from a group consisting of silicon, glass, polycarbonate, polydimethylsiloxane, and polymethylmetaacrylate. The first substrate may be etched to have a desired thickness by wet etching or dry etching such as a reactive ion etching. If necessary, the photolithographic process and the etching process may be repeated several times to allow the flow path and the chamber to have varying depths. A hydrophobic treatment is applied to the upper portion of a second substrate, which is a cover for preventing evaporation of the DNA reaction fluid in the PCR chamber to resist wetting. After patterns of an inlet and an outlet for the sample are formed on the first substrate through photolithography, the inlet and the outlet are finished by sound-blasting. If it is necessary to form an electrode structure on the second substrate, electrode patterns are formed through photolithography and are obtained using a lift-off procedure. Subsequently, the first and second substrates are bonded using a method such as anodic bonding, fluorine bonding, thermal bonding, or polymer film bonding.

According to still another aspect of the present invention, there is provided a lab-on-a-chip comprising any one of the above-described polymerase chain reaction apparatuses and an electrophoresis performing unit connected to the polymerase chain reaction apparatus in a fluidic manner.

When the sample in the chip passes through the PCR apparatus, the DNA is amplified. When the sample passes through the electrophoresis performing unit, the DNA is separated depending on its molecular amount or charge to detect target DNA.

The substrates may be made of a material selected from a group consisting of glass, quartz, silicon, plastic, polymer, ceramic, or metal. The electrophoresis unit may have multiple channels performing capillary electrophoresis. The PCR amplification apparatus and the electrophoresis performing unit may be formed on a substrate through photolithography.

According to still another aspect of the present invention, there is provided an inkjet spotter comprising: any one of the above-described polymerase chain reaction apparatuses formed on a substrate; a restrictor connected to the polymerase chain reaction apparatus in a fluidic manner; an ejecting chamber storing a DNA solution from the polymerase chain reaction apparatus via the restrictor; an ejecting driving element providing a driving force of the DNA solution ejection; and a nozzle ejecting the DNA solution from the ejecting chamber.

The inkjet spotter according to the present invention may be similar to a typical inkjet spotter used to manufacture a conventional DNA micro-array except for a PCT apparatus. The ejecting driving element may be a thermal type (similar to that disclosed in U.S Pat. No. 4,438,191), a Piezo type (similar to that disclosed in U.S. Pat. No. 5,748,214), or an electric field type (similar to that disclosed in U.S. Pat. No. 4,752,783).

Hereinafter, exemplary embodiments according to the present invention will be described in detail with reference to the accompanying drawings.

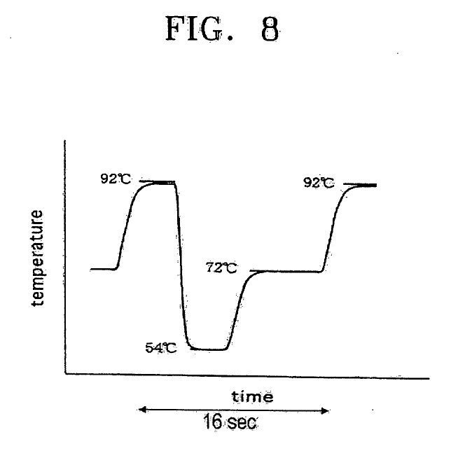

FIG. 6 is a schematic diagram of a PCR apparatus using Marangoni convection according to an embodiment of the present invention. The PCR apparatus includes a substrate 10, a high-temperature sidewall 1 erected on the substrate 10, a low-temperature sidewall 2 erected on the substrate 10 and facing the high-temperature sidewall 1, and a reaction chamber 3 consisting of the substrate 10, the high-temperature sidewall 1, and the low-temperature sidewall 2. A fluid contained in the reaction chamber 3 is thermally circulated as indicated by arrows in FIG. 6 between the high-temperature sidewall 1 and the low-temperature sidewall 2 due to Marangoni convection caused by a surface tension gradient resulting from a temperature difference in an interface between the fluid and air. In other words, the Marangoni convection flow can be induced between the high-temperature sidewall 1 and the low-temperature sidewall 2 by forming an interface between the fluid and the air from the high-temperature sidewall 1 to the low-temperature sidewall 2. The induced flow results in circulation of the fluid in the chamber, which results in the PCR amplification. The PCR apparatus according to the present embodiment may further include a cover 20 over the reaction chamber 3.

The Marangoni convection flow occurs from the high-temperature sidewall 1, which has a temperature of about 95° C., toward the low-temperature sidewall 2, which has a temperature of about 50° C. The DNA sample contained in the fluid is denatured at about 94° C. near the high-temperature sidewall 1, flows along the interface between the fluid and air toward the low-temperature sidewall 2, and annealed at about 55° C. near the low-temperature sidewall 2. Accordingly, the DNA sample flows along the bottom of the chamber, either adiabatically or at a constant temperature of 72° C. to generate extension. The denaturing, annealing, and extension constitute a thermal cycle. According to the present invention, the time required to complete one thermal cycle can be as low as 5 seconds or less. However, an excessively short cycle may result in insufficient DNA amplification. Therefore, an appropriate time period for one cycle is within a range from 5 seconds to 30 seconds. The time for performing a cycle can be adjusted by controlling the size of the reaction chamber and the surface tension of the reaction fluid using an electric field on a surface active agent.

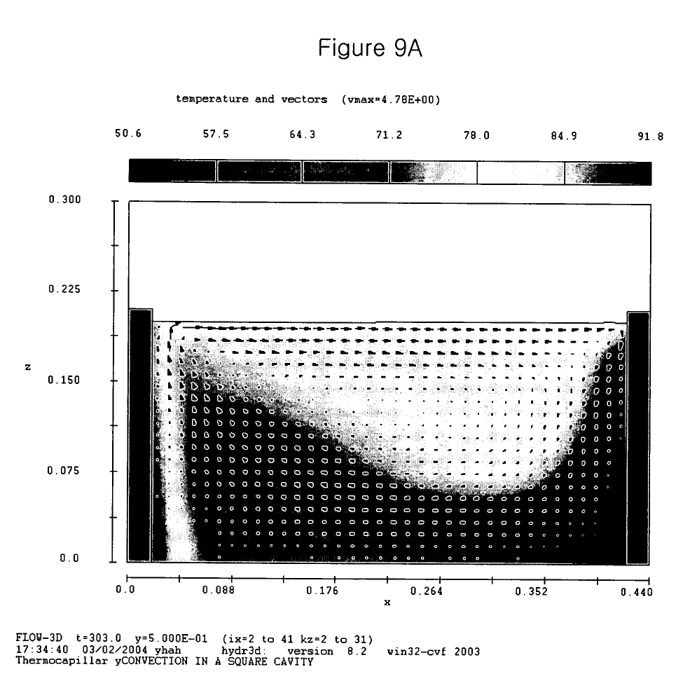

FIG. 7 is a schematic diagram illustrating a principle of generating thermal capillary flow depending on a surface tension gradient at an interface between a fluid and air. The Marangoni convection is based on the fact that the surface tension (s) is a function of temperature. If there is a temperature gradient along the interface between the reaction fluid and the air, the surface tension is lower in a high temperature region than a low temperature region. As a result, a surface tension gradient is generated, and thus the fluid flows from the the high temperature region to the low temperature region, thus generating the Marangoni convection in the reaction chamber.

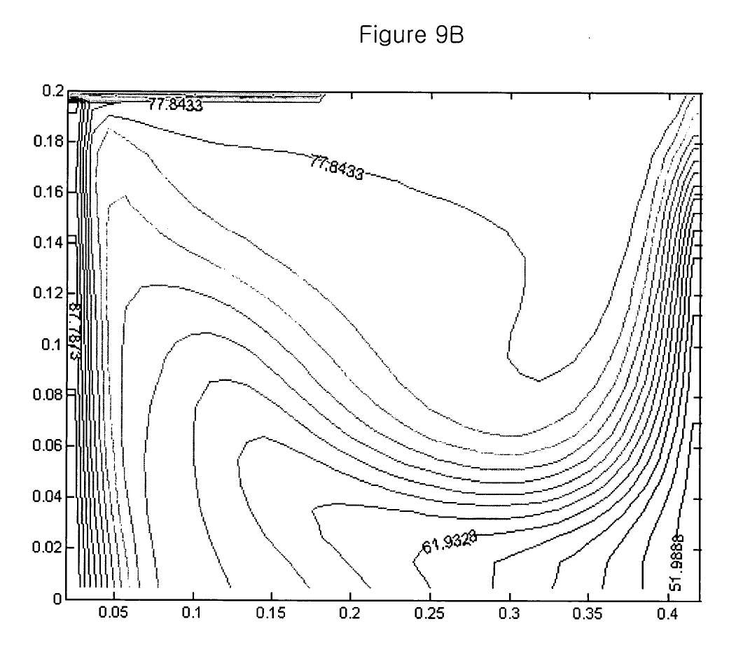

FIG. 8 is a temperature vs. time graph for the PCR reaction according to an embodiment of the present invention. The graph shows the temperature change of the sample circulating in the reaction chamber according to time. In the PCR apparatus according to an embodiment of the present invention, the sample is heated to a temperature of about 94° C. near the high-temperature sidewall 1 to generate DNA denaturing, and quickly flows to the low-temperature sidewall 2 along an upper region of the reaction fluid due to the Marangoni convection. Near the low-temperature sidewall 2, the temperature of the sample is reduced to about 55° C., and the DNA in the sample is annealed. Then, the sample flows from the low-temperature sidewall 2 to the high-temperature sidewall 1 along the lower region of the reaction fluid, and experiences extension at a temperature of about 72° C. A time period for completing such a PCR cycle is about 8 seconds.

A method of manufacturing the PCR apparatus will now be described.

First, a flow path and a PCR chamber pattern are formed on a first substrate through photolithography. The first substrate may be made of a material selected from a group consisting of silicon, glass, polycarbonate, polydimethylsiloxane, and polymethylmetaacrylate. Wet etching or dry etching such as reactive ion etching may be used to form the first substrate to a desired thickness. If required, the photolithographic process and the etching process may be repeated several times to provide the flow path and the chamber with varying depths. In the case of a silicon substrate, a silicon oxide film, which will be used as a DNA absorption protection film as well as an electric insulation film, having a thickness of several hundreds of nanometers, is deposited by wet etching after the final etching process. Electrodes on the bottom of the first substrate are patterned using photolithography, a thin film made of platinum, tantal aluminum, or polysilicon, which will be used as a thin film heater, is coated thereon, and then the first electrodes are completed by performing a lift-off procedure. Then, a hydrophobic treatment is applied to the upper surface of a second substrate, which is a cover for preventing evaporation of the DNA reaction fluid from the PCR chamber, to resist wetting. After the pattern of the inlet and outlet for the sample are formed in the first substrate through photolithography, the first substrate is sand-blasted to finish the inlet and outlet. If it is necessary to form an electrode structure on the second substrate, the electrode patterns are formed through photolithography and performing a lift-off procedure. Subsequently, the first and second substrates are bonded using a method such as anodic bonding, fluorine bonding, thermal bonding, or polymer film bonding.

The present invention will now be fully described using examples. The examples should be considered in descriptive sense only and are not for purposes of limitation. Therefore, the scope of the invention is not defined by the following embodiments.

### EXAMPLE 1

### Two-dimensional Analysis

Thermal-flow fields in the Marangoni PCR chamber have been analyzed using a commercial professional numerical analysis tool, FLOW3D (www.flow3d.com), specialized for a surface flow analysis. In the analysis, it was assumed that the sidewalls were maintained at temperatures of 95° C. and 50° C., respectively. Also, it was assumed that a buffer solution had a thermal conductivity of 0.656 W/m K, a specific heat of 4187 J/Kg K, and a surface tension coefficient of 72 dyne/cm. It was also assumed that the buffer solution had a contact angle of 90° by supposing that a hydrophobic treatment was used. Further, a surface tension coefficient based on the temperature was 0.16 dyne/cm K, corresponding to that of water.

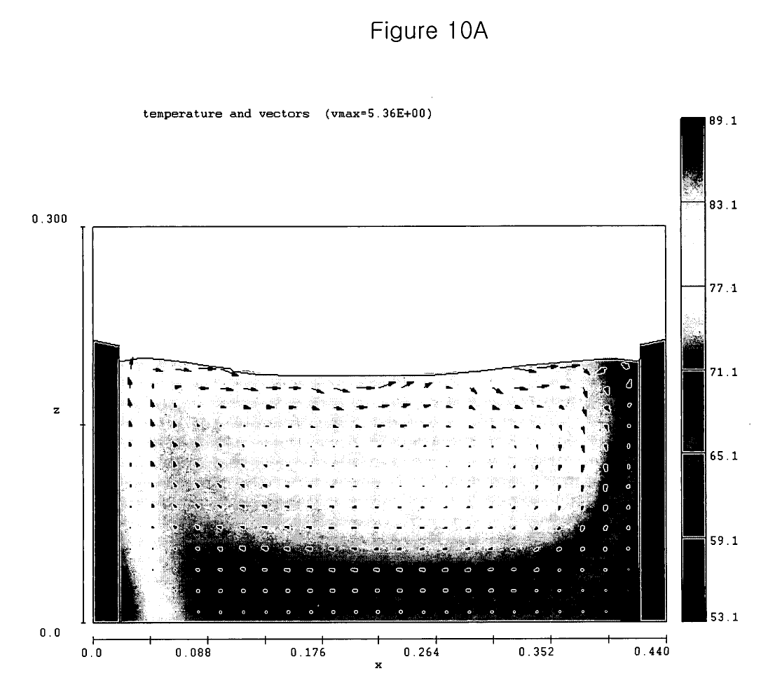

FIG. 9 illustrates the result of a Marangoni flow analysis in a two-dimensional container having a width of 4 mm. This result is based on the assumption that the fluid is divided into 40×30 grids in x and y directions. In this case, the widths of the sidewalls are negligible because their lengths are much greater than the widths. A temperature gradient is not generated along the width of the container. The highest speed of the fluid at an interface is about 4˜5 cm/s. Since the fluid originating from the high-temperature sidewall is returned to its position after about 8 sec, one cycle of PCR amplification can be accomplished in a very short time. FIG. 8 shows temperature according to time in each cycle of the PCR. A cycle includes a 1-second denaturing process, a 2-second annealing process, and a 2-second extension process. Considering the transient times between each process, each cycle of the PCR amplification takes about 8 seconds.

### EXAMPLE 2

### Three-dimensional Analysis

Similar to the Example 1, thermal-flow fields in the Marangoni PCR chamber have been analyzed by using FLOW3D. It was assumed that the fluid was divided into 25×25×20 grids in x, y, and z directions. Also, it was assumed that the conditions such as boundary conditions and material properties were similar to those of the two-dimensional analysis.

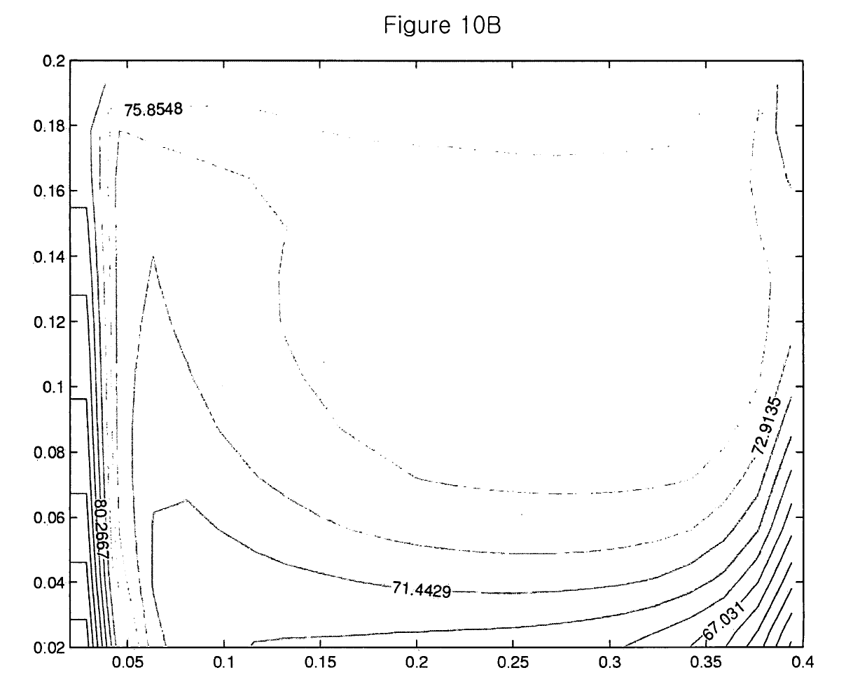

FIG. 10 illustrates the result of Marangoni flow analysis in a three-dimensional container having a width of 4 mm. Since the length is nearly equal to the width in an actual PCR reactor, it was assumed that the Marangoni flow is generated in a three-dimensional rectangular container having a length of 4 mm, a width of 4 mm, and a height of 2 mm. Similarly, it was assumed that a temperature gradient is generated along only the length. Both the sidewalls along the length of the container were in adiabatic conditions. As a result, it was confirmed that Marangoni convection having a high speed of 4˜5 cm/s exists at an interface between the fluid and air and there is no significant difference between the three-dimensional analysis and the two-dimensional analysis. Also, it was confirmed that one PCR cycle requires about 8˜10 seconds and 30 cycles requires about 4˜5 minutes. In comparison with a conventional cyclic temperature control type PCR apparatus, in which it takes 15 seconds for only the cooling and 30 minutes or more for 30 cycles, a PCR apparatus according to the present invention is quite advantageous.

### EXAMPLE 3

### Inkjet Spotter Capable of Marangoni PCR DNA Amplification

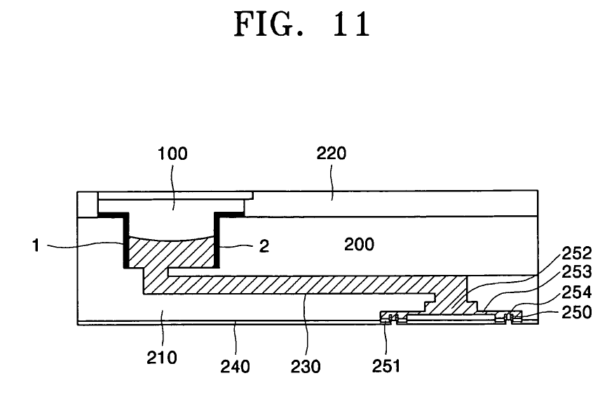

An inkjet spotter having a PCR apparatus according to an embodiment of the present invention is manufactured. FIG. 11 illustrates an exemplary thermal type inkjet spotter according to an embodiment of the present invention. Referring to FIG. 11, the inkjet spotter includes a typical spotter 200 and a Marangoni PCR apparatus 100. The Marangoni PCR apparatus 100 includes a high-temperature sidewall 1 and a low-temperature sidewall 2 erected on a substrate 210, and the spotter 200 is connected to the Marangoni PCR apparatus via a micro-channel 230. The spotter 200 includes a manifold 252 for supplying a DNA solution to a plurality of ejecting chambers, a restrictor 253 serving as a guide to the ejecting chamber, an ejecting chamber 254 for storing the DNA solution before ejecting it, a thin film heater 251 serving as an ejecting driver, and a nozzle 250 for ejecting the amplified DNA solution. The inkjet spotter 200 can be used to spot a desired amount of the PCR amplified DNA solution using Marangoni convection in desired positions. The inkjet spotter according to the present embodiment may adopt one of various ejecting driving methods such as a thermal method, a Piezo method, or an electric field method.

In the conventional PCR amplification, DNA amplification was accomplished by cyclically heating and cooling a chamber containing a DNA buffer solution. Therefore, it was difficult to control temperature, there was high power consumption, and it takes a long time to complete a cycle of amplification. However, according to the present invention, the PCR amplification is automatically accomplished by a surface tension flow generated by Marangoni convection resulting from a temperature difference in an interface between a fluid and air when sidewalls of the chamber are maintained at a predetermined temperature difference. As a result, it is possible to reduce power consumption, simplify the configuration of a temperature control circuit, and reduce the time for an amplification cycle.

While the present invention has been particularly shown and described with reference to exemplary embodiments thereof, it will be understood by those skilled in the art that various changes in form and details may be made therein without departing from the spirit and scope of the invention as defined by the appended claims. The exemplary embodiments should be considered in descriptive sense only and not for purposes of limitation. Therefore, the scope of the invention is defined not by the detailed description of the invention but by the appended claims, and all differences within the scope will be construed as being included in the present invention.

1. A polymerase chain reaction apparatus comprising:
a substrate;
a high-temperature sidewall erected on the substrate;
a low-temperature sidewall erected on the substrate and facing the high-temperature sidewall; and
a reaction chamber consisting of the substrate, the high-temperature sidewall, and the low-temperature sidewall,
wherein a sample contained in the reaction chamber is repetitively thermal-circulated between the high-temperature sidewall and the low-temperature sidewall using Marangoni convection generated by a surface tension gradient resulting from a temperature difference in an interface between the sample and air.

2. The polymerase chain reaction apparatus according to claim 1, further comprising a cover for covering the reaction chamber.

3. The polymerase chain reaction apparatus according to claim 1, wherein the high-temperature sidewall is heated by a heater to a constant temperature of 92to 97° C.

4. The polymerase chain reaction apparatus according to claim 3, wherein the heater is a thin film heater made of a material selected from a group consisting of Pt, poly-silicon, and tantal aluminum.

5. The polymerase chain reaction apparatus according to claim 1, wherein the low-temperature sidewall is cooled by a cooler to a constant temperature of 44to 56° C.

6. The polymerase chain reaction apparatus according to claim 5, wherein the cooler is a cooling fan, a heat exchanger, or a Peltier device.

7. The polymerase chain reaction apparatus according to claim 1, wherein a gap between the high-temperature sidewall and the low-temperature sidewall is 2 mm to 3 cm.

8. The polymerase chain reaction apparatus according to claim 1, wherein the reaction chamber is made of a material selected from a group consisting of glass, quartz, silicon, plastic, polymer, ceramic, and metal.

9. The polymerase chain reaction apparatus according to claim 1, wherein the reaction chamber has an optical detection window.

10. A polymerase chain reaction method comprising:
putting a sample into the reaction chamber of the polymerase chain reaction apparatus according to claim 1,
maintaining temperatures of the high-temperature sidewall and the low-temperature sidewall constant; and
repetitively thermal-circulating the sample contained in the reaction chamber between the high-temperature sidewall and the low-temperature sidewall using Marangoni convection.

11. The polymerase chain reaction method according to claim 10, wherein the high-temperature sidewall is maintained at a constant temperature of 92to 97° C., and the low-temperature sidewall is maintained at a constant temperature of 48to 54° C.

12. The polymerase chain reaction method according to claim 10, wherein the sample contains a fluorescent material to detect the amount of amplification of a nucleic acid in a real-time manner.

13. A method of manufacturing a polymerase chain reaction apparatus, comprising a photolithographic process, a wet etching process or a dry etching process such as a reactive ion etching, and a hydrophobic treatment process of a reactor cover.

14. A lab-on-a-chip comprising the polymerase chain reaction apparatus according to claim 1 and an electrophoresis performing unit connected to the polymerase chain reaction apparatus in a fluidic manner.

15. An inkjet spotter comprising:
the polymerase chain reaction apparatus according to claim 1 formed on a substrate;
a restrictor connected to the polymerase chain reaction apparatus in a fluidic manner;
an ejecting chamber storing a DNA solution from the polymerase chain reaction apparatus via the restrictor;
an ejecting driving element providing a driving force of the DNA solution ejection; and
a nozzle ejecting the DNA solution from the ejecting chamber.
